(19) United States
(12) Patent Application Publication
(10) Pub. No.: US 2017/0335152 A1
(43) Pub. Date: Nov. 23, 2017

(54) IN-LINE POLYOLEFIN BASED ADHESIVE COMPOSITIONS HAVING GRAFT POLYOLEFIN/ELASTOMER COPOLYMERS

(71) Applicant: EQUISTAR CHEMICALS, LP, Houston, TX (US)

(72) Inventor: MAGED G. BOTROS, LIBERTY TOWNSHIP, OH (US)

(73) Assignee: EQUISTAR CHEMICALS, LP, HOUSTON, TX (US)

(21) Appl. No.: 15/593,600

(22) Filed: May 12, 2017

Related U.S. Application Data
(60) Provisional application No. 62/413,196, filed on Oct. 26, 2016.
(60) Provisional application No. 62/339,247, filed on May 20, 2016.

(51) Int. Cl. C09J 151/00; C08F 4/649; C08F 4/12; C08F 4/02; C07D 309/10; C08L 29/00; C07D 307/935

(52) U.S. Cl. CPC C09J 151/006; C08L 29/00; C08F 4/6494; C07D 307/935; C08F 4/022; C07D 309/10; C08F 4/12

(57) ABSTRACT

The present disclosure relates to adhesive compositions, processes of forming adhesive compositions, and multi-layer films. The processes generally include contacting an olefin monomer with a catalyst system within a polymerization zone to form an olefin based polymer under polymerization conditions sufficient to form the olefin based polymer, the catalyst system including a metal component generally represented by the formula:

MRₓ;

wherein M is a transition metal, R is a halogen, an alkoxy, or a hydrocarboxyl group and x is the valence of the transition metal, wherein the catalyst system further includes an internal donor (ID) comprising a C₃-C₆ cyclic ether; and withdrawing the olefin based polymer from the polymerization zone; and melt blending the olefin based polymer with a graft (polyolefin/elastomer) copolymer to form a polyolefin based adhesive composition, wherein the process is an in-line process.

### CROSS-REFERENCE TO RELATED APPLICATIONS

This application is the Non-Provisional Patent Application, which claims benefit of priority to U.S. Provisional Application No. 62/413,196, filed Oct. 26, 2016 and U.S. Provisional Application No. 62/339,247, filed on May 20, 2016, the contents of which are incorporated herein by reference in their entirety.

### STATEMENT REGARDING FEDERALLY SPONSORED RESEARCH OR DEVELOPMENT

Not applicable.

### FIELD OF THE INVENTION

Embodiments of the present disclosure generally relate to polyolefin based adhesive compositions.

### BACKGROUND OF THE INVENTION

This section introduces information that may be related to or provide context for some aspects of the techniques described herein and/or claimed below. This information is background for facilitating a better understanding of that which is disclosed herein. Such background may include a discussion of “related” art. That such art is related in no way implies that it is also “prior” art. The related art may or may not be prior art. The discussion is to be read in this light, and not as an admission of prior art.

Multi-layer films are widely used in a variety of applications, including packaging applications. Depending on the intended end-use application, the number and arrangement of layers and type of resin employed in each layer will vary.

One challenge experienced in the fabrication of multi-layer films is achieving sufficient bond strength between the various layers of the multi-layer film. In order to improve bonding between layers, a tie-layer may be disposed between one or more layers of the multi-layer film. However, even when multi-layer films include tie-layers, difficulties in adhering dissimilar layers can occur. Thus, it is desirable to develop adhesive compositions for use in tie-layers that are capable of sufficiently adhering dissimilar layers within a multi-layer film.

### SUMMARY OF THE INVENTION

Various embodiments of the technology described herein are directed to resolving, or at least reducing, one or more of the problems mentioned above. Some embodiments of the technology include processes of forming adhesive compositions. The processes generally include contacting an olefin monomer with a catalyst system within a polymerization zone to form an olefin based polymer under polymerization conditions sufficient to form the olefin based polymer, the catalyst system including a metal component generally represented by the formula:

MRₓ;

wherein M is a transition metal, R is a halogen, an alkoxy, or a hydrocarboxyl group and x is the valence of the transition metal, wherein the catalyst system further includes an internal donor (ID) comprising a C₃-C₆ cyclic ether; and withdrawing the olefin based polymer from the polymerization zone; and melt blending the olefin based polymer with a graft (polyolefin/elastomer) copolymer to form a polyolefin based adhesive composition, wherein the process is an in-line process.

One or more embodiments include the process of the preceding paragraph, wherein the olefin based polymer contacts the graft (polyolefin/elastomer) copolymer prior to pelletization of the olefin based polymer.

One or more embodiments include the process of any preceding paragraph and further include melt blending the olefin based polymer and the graft (polyolefin/elastomer) copolymer in the presence of an adhesion promoting additive.

One or more embodiments include the process of any preceding paragraph, wherein the transition metal is selected from titanium, chromium and vanadium.

One or more embodiments include the process of any preceding paragraph, wherein the metal component is selected from TiCl₄, TiBr₄, Ti(OC₂H₅)₃Cl, Ti(OC₃H₇)₂Cl₂, Ti(OC₆H₁₃)₂Cl₂, Ti(OC₂H₅)₂Br₂ and Ti(OC₁₂H₂₅)Cl₃.

One or more embodiments include the process of any preceding paragraph, wherein the catalyst system further includes an organoaluminum compound selected from trimethyl aluminum (TMA), triethyl aluminum (TEAl) and triisobutyl aluminum (TiBAl).

One or more embodiments include the process of any preceding paragraph, wherein the cyclic ethers are selected from tetrahydrofuran, dioxane, methyltetrahydrofuran and combinations thereof.

One or more embodiments include the process of any preceding paragraph, wherein the catalyst system further includes a support material including a magnesium halide.

One or more embodiments include the process of any preceding paragraph, wherein the catalyst system exhibits a molar ratio Mg:Ti of greater than 5:1.

One or more embodiments include the process of any preceding paragraph, wherein the catalyst system exhibits a molar ratio Mg:ID of less than 3:1.

One or more embodiments include the process of any preceding paragraph, wherein the olefin based polymer exhibits a density (determined in accordance with ASTM D-792) of from 0.86 g/cm³ to 0.94 g/cm³.

One or more embodiments include the process of any preceding paragraph, wherein the olefin based polymer exhibits a melt index (MI₂) (determined in accordance with ASTM D-1238) in a range of 0.1 dg/min to 15 dg/min.

One or more embodiments include the process of any preceding paragraph, wherein the polyolefin based adhesive composition includes the graft (polyolefin/elastomer) copolymer in a range of 0.5 wt. % to 50 wt. %, based on the total weight of the polyolefin based adhesive composition.

One or more embodiments include an adhesive composition including a polyolefin based adhesive composition formed with a single heat cycle and including an olefin based polymer formed with catalyst system including a metal component generally represented by the formula:

MRₓ;

wherein M is a transition metal, R is a halogen, an alkoxy, or a hydrocarboxyl group and x is the valence of the transition metal, wherein the catalyst system further includes an internal donor including a C₃-C₆ cyclic ether; and supported on MgCl_{2;} and a graft (polyolefin/elastomer) copolymer.

One or more embodiments include the adhesive composition of the preceding paragraph exhibiting a lower gel count and lower yellowness index than an identical composition formed via an off-line process.

One or more embodiments include the adhesive composition of any preceding paragraph, wherein the cyclic ethers are selected from tetrahydrofuran, dioxane, methyltetrahydrofuran and combinations thereof.

One or more embodiments include a multi-layer film including a plurality of resin layers; and one or more tie-layers disposed between at least two of the resin layers, wherein the tie layers are formed of the adhesive composition of any preceding paragraph.

While multiple embodiments are disclosed, still other embodiments will become apparent to those skilled in the art from the following detailed description. As will be apparent, certain embodiments, as disclosed herein, are capable of modifications in various obvious aspects, all without departing from the spirit and scope of the claims as presented herein. Accordingly, the drawings and detailed description are to be regarded as illustrative in nature and not restrictive.

### BRIEF DESCRIPTION OF DRAWINGS

The claimed subject matter may be understood by reference to the following description taken in conjunction with the accompanying drawings, in which like reference numerals identify like elements, and in which:

FIG. 1 illustrates an embodiment of an in-line process of forming an adhesive composition.

While the claimed subject matter is susceptible to various modifications and alternative forms, the drawings illustrate specific embodiments herein described in detail by way of example. It should be understood, however, that the description herein of specific embodiments of the technology is not intended to limit the claimed subject matter to the particular forms disclosed, but on the contrary, the intention is to cover all modifications, equivalents, and alternatives falling within the spirit and scope as defined by the appended claims.

### DETAILED DESCRIPTION OF THE INVENTION

Illustrative embodiments of the subject matter claimed below will now be disclosed. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The embodiments illustratively disclosed herein may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. Further, various ranges and/or numerical limitations may be expressly stated below. It should be recognized that unless stated otherwise, it is intended that endpoints are to be interchangeable. Further, any ranges include iterative ranges of similar magnitude falling within the expressly stated ranges or limitations disclosed herein is to be understood to set forth every number and range encompassed within the broader range of values. It is to be noted that the terms “range” and “ranging” as used herein generally refer to a value within a specified range and encompass all values within that entire specified range.

Furthermore, various modifications may be made within the scope of the disclosure as herein intended, and embodiments of the disclosure may include combinations of features other than those expressly claimed.

Various terms as used herein are shown below. To the extent a term used in a claim is not defined below, it should be given the broadest definition skilled persons in the pertinent art have given that term as reflected in printed publications and issued patents at the time of filing. Further, unless otherwise specified, all compounds described herein may be substituted or unsubstituted and the listing of compounds includes derivatives thereof.

Further, various ranges and/or numerical limitations may be expressly stated below. It should be recognized that unless stated otherwise, it is intended that endpoints are to be interchangeable. Further, any ranges include iterative ranges of similar magnitude falling within the expressly stated ranges or limitations.

Polyolefin based adhesive compositions and methods of making and using the same are described herein. The polyolefin based adhesive compositions are generally formed of an olefin based polymer and a graft (polyolefin/elastomer) copolymer.

Graft (polyolefin/elastomer) copolymers as used herein are a copolymer of (i) a grafted polyolefin and (ii) an olefin elastomer, wherein the copolymer is produced by a radical coupling reaction between a live, grafted polyolefin and the olefin elastomer. Live, grafted polyolefins suitable for use in making the graft compositions are manufactured by reacting polyolefins with unsaturated monomers at elevated temperatures, with or without a free-radical initiator, under conditions effective to graft unsaturated monomer units onto the polyolefin backbone.

Polyolefins suitable for making the live, grafted polyolefins include high density polyethylenes (HDPE), medium density polyethylenes (HDPE), low density polyethylenes (LDPE), linear low density polyethylenes (LLDPE), polypropylenes, ethylene-propylene copolymers, impact-modified polypropylenes, and the like, and blends thereof. In some embodiments, polyolefins for making the grafted polyolefin are polyethylenes such as HDPE and LLDPE. As used herein, the term “high density polyethylene” or HDPE refers to ethylene based polymers having a density of from about 0.94 g/cm³ to about 0.97g/cm³, for example.

An unsaturated monomer reacts with the polyolefin to produce the grafted polyolefin. Suitable unsaturated monomers are well known and may include ethylenically unsaturated carboxylic acids and acid derivatives such as esters, anhydrides, acid salts, and the like. Examples include acrylic acid, methacrylic acid, maleic acid, fumaric acid, itaconic acid, citraconic acid, maleic anhydride, tetrahydrophthalic anhydride, norborn-5-ene-2,3-dicarboxylic acid anhydride, nadic anhydride, himic anhydride, and the like, and mixtures thereof. Other suitable unsaturated monomers are described in U.S. Pat. Appl. Publ. Nos. 2004/0097637 and 2007/0054142, the teachings of which are incorporated herein by reference.

The relative amounts of unsaturated monomer and polyolefin used will vary and depend on factors such as the nature of the polyolefin and unsaturated monomer, reaction conditions, available equipment, and other factors. Usually, the unsaturated monomer is used in an amount within the range of 0.1 to 15 wt. %, from 0.5 to 6 wt. %, and from 1 to 3 wt. %, based on the amount of live, grafted polyolefin produced.

Grafting is accomplished according to known procedures, generally by heating a mixture of the polyolefin and unsaturated monomer(s). In some embodiments, the grafted polyolefin is prepared by melt blending the polyolefin with the unsaturated monomer in a shear-imparting extruder/reactor. Twin screw extruders such as those marketed by Coperion under the designations ZSK-53, ZSK-83, ZSK-90 and ZSK-92 are especially useful for performing the grafting step. A free-radical initiator such as an organic peroxide can optionally be employed.

Grafting of the unsaturated monomer and polyolefin to generate the live, grafted polyolefin is performed at elevated temperatures, for instance within the range of 180° C. to 400° C., from 200° C. to 375° C., and from 230° C. to 350° C. Shear rates in the extruder can vary over a wide range, such as from 30 to 1000 rpm, from 100 to 600 rpm, and from 200 to 400 rpm.

As used herein, a “live, grafted polyolefin,” refers to a grafted polyolefin that can further react with added olefin elastomer and any residual polyolefin, unsaturated monomer, and/or free-radical initiator used to make the grafted polyolefin. Commercially available grafted polyolefins are not “live” because the free-radical content has fully reacted or has been quenched during workup of the product, for instance during pelletization. A live, grafted polyolefin contains active free-radical species generated thermally by visbreaking or from peroxide decomposition. The residual radical content allows reaction to continue upon combination of the freshly made grafted polyolefin, including while the polyolefin is still molten, with an added olefin elastomer. One or more of the grafted polyolefin, olefin elastomer, residual polyolefin, and residual unsaturated monomer may be involved in a secondary reaction.

Thus, in the second process step for making the graft composition, the live, grafted polyolefin (and any residual polyolefin and/or unsaturated monomer) may be reacted with an olefin elastomer. This reaction can be performed using any suitable reactor. In some embodiments, the reaction is performed by combining the freshly prepared live, grafted polyolefin with the olefin elastomer in a shear-imparting extruder/reactor as described earlier. In one embodiment, the live, grafted polyolefin is transferred while still molten from an outlet of a first extruder directly to a second extruder in which a reaction with the olefin elastomer occurs.

The amount of olefin elastomer used depends on the nature of the elastomer and grafted polyolefin, the desired tie-layer properties, reaction conditions, equipment, and other factors. Generally, however, the amount of elastomer used will be in the range of 5 to 60 wt. %, from 20 to 50 wt. %, and from 30 to 40 wt. %, based on the amount of graft composition produced.

The live, grafted polyolefin and the olefin elastomer react at elevated temperature, such as at temperatures within the range of 120° C. to 300° C., from 135° C. to 260° C., and from 150° C. to 230° C. In some embodiments, the temperature for the reaction used to make this graft composition is lower than that used to make the live, grafted polyolefin. Shear rates in the extruder for this step can vary over a wide range, including from 30 to 1000 rpm, from 100 to 600 rpm, and from 200 to 400 rpm.

The resulting grafted [polyolefin/elastomer] composition is conveniently quenched and pelletized at this point, but it can be combined immediately after preparation with base resin as further described below.

Suitable olefin elastomers include ethylene-propylene rubber (EPR), ethylene-propylene-diene monomer rubber (EPDM), the like, and mixtures thereof. As used herein, “elastomer” refers to products having rubber-like properties and little or no crystallinity. In some embodiments, the olefin elastomers contain from 10 to 80 wt. % of ethylene recurring units, including from 10 to 70 wt. % of ethylene units. Commercially available olefin elastomers include Lanxess Corporation's Buna® EP T2070 (68% ethylene, 32% propylene); Buna EP T2370 (3% ethylidene norbornene, 72% ethylene, 25% propylene); Buna EP T2460 (4% ethylidene norbornene, 62% ethylene, and 34% propylene); ExxonMobil Chemical's Vistalon® 707 (72% ethylene, 28% propylene); Vistalon 722 (72% ethylene, 28% propylene); and Vistalon 828 (60% ethylene, 40% propylene). Suitable ethylene-propylene elastomers also include ExxonMobil Chemical's Vistamaxx® elastomers, including grades 6100, 1100, and 3000, and Dow Chemical's Versify® elastomers, including grades DP3200.01, DP3300.01, and DP3400.01, which have ethylene contents of 9, 12, and 15 wt %, respectively. Additional EPDM rubbers include Dow's Nordel™ hydrocarbon rubber, e.g., the 3722P, 4760P, and 4770R grades. Suitable graft (polyolefin/elastomer) copolymer is further described in U.S. Pat. No. 8,637,159, the disclosure of which is incorporated herein by reference.

Prior processes (off-line systems) for forming polyolefin based adhesive compositions generally included extruding olefin based polymers upon withdrawal from a polymerization zone to form polyolefin pellets in a first extrusion process and then contacting those polyolefin pellets with a functionalized polyolefin in a second (or subsequent) extrusion process to form the polyolefin based adhesive composition.

Each extrusion process is generally referred to herein as a “heat cycle.” A heat cycle generally refers to heating a respective polymer to a temperature sufficient to at least partially melt the polymer and form a molten polymer, and then cooling the molten polymer to a temperature sufficient to at least partially solidify the molten polymer.

In contrast, in the embodiments described herein, the olefin based polymer undergoes a single heat cycle in the formation of the polyolefin based adhesive composition. For example, in one or more embodiments, the olefin based polymer recovered from a polymerization zone is directly contacted with the graft (polyolefin/elastomer) copolymer to form the polyolefin based adhesive composition. For example, olefin based polymer may be withdrawn from the polymerization zone and melt blended with the graft (polyolefin/elastomer) copolymer to form the polyolefin based adhesive composition. Such melt blending may occur via extrusion, for example. In such embodiments, it is to be noted that while the olefin based polymer contacts the graft (polyolefin/elastomer) copolymer during the melt blending, the initial contact of the olefin based polymer and the graft (polyolefin/elastomer) copolymer may occur prior to melt blending, such as in a mixer, a feeder or a storage vessel, for example.

As used herein, the term “directly” refers to withdrawing the olefin based polymer from the polymerization zone and contacting the olefin based polymer with the graft (polyolefin/elastomer) copolymer without an intervening heat cycle. In such embodiments, the olefin based polymer contacts the graft (polyolefin/elastomer) copolymer prior to pelletization of the olefin based polymer and thus, the olefin based polymer undergoes a single heat cycle in the formation of the polyolefin based adhesive composition.

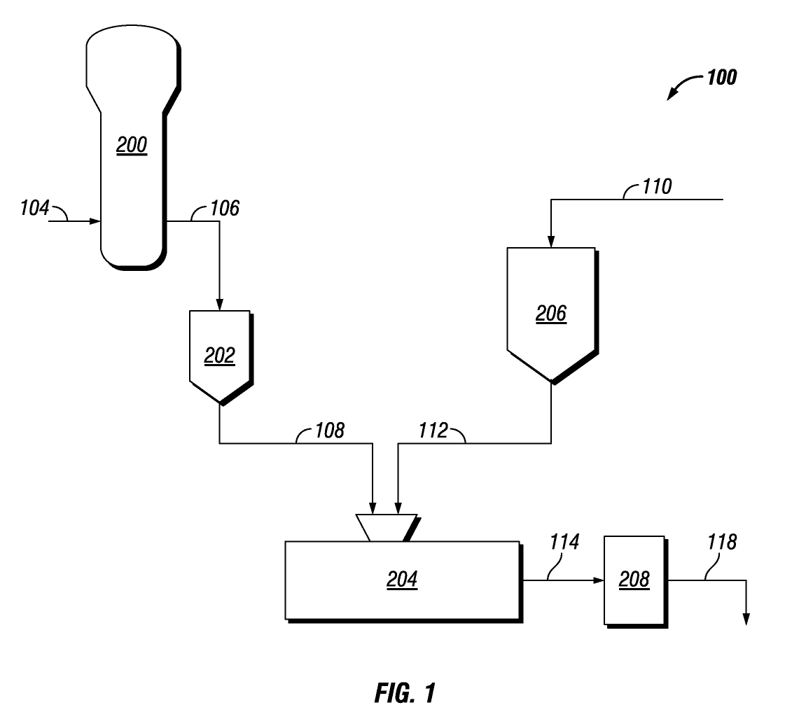

An illustrative schematic of such an embodiment is illustrated in FIG. 1, which illustrates in-line process 100 for forming an adhesive composition. Within the process 100, olefin monomer (not shown) and optionally co-monomer (not shown) is introduced into a polymerization zone (or reactor) 200 via a reactor feed line 104. The olefin monomer contacts a polymerization catalyst system (not shown) disposed within the polymerization zone 200 under polymerization conditions sufficient to form an olefin based polymer (not shown). The olefin based polymer (not shown) is withdrawn or recovered from the polymerization zone 200 via reactor exit line 106 and passes through an optional powder silo (vessel or bin) 202 to an extruder 204 via first an extruder-feed line 108. A graft (polyolefin/elastomer) copolymer (not shown) is introduced into an optional graft silo (vessel or bin) 206 via graft-feed line 110. The graft (polyolefin/elastomer) copolymer (not shown) is fed to the extruder 204 via a second extruder-feed line 112. The graft (polyolefin/elastomer) copolymer (not shown) and the olefin based polymer (not shown) are mixed in the extruder 204 (optionally under shear mixing sufficient to blend the components and any additives). The mixed graft (polyolefin/elastomer) copolymer polyolefin and olefin based polymer form the polyolefin based adhesive composition (not shown) within the extruder 204. The adhesive composition (not shown) is fed (optionally by an un-shown melt pump) from the extruder 204 to a pelletizer 208 via a pelletizer feed line 114. The adhesive composition (not shown) is pelletized in the pelletizer 208 and recovered as product via product line 118. Optionally, the pelletized adhesive composition may be accumulated in bins (not shown) and shipped to customers. Additional equipment components, such as feeders, additive bins, degassers, screen packs, and storage tanks are contemplated for use but are known in the art and thus not shown in FIG. 1.

In an embodiment, the processes described herein are in-line processes to form adhesive resins (also called adhesive compositions). In an embodiment, an in-line process is a process in which an adhesive resin is formed using a polyolefin from a reactor (also called the olefin based polymer) that undergoes a single heat cycle (or a single heat history, or a single pelletization step). In an embodiment, the in-line process includes withdrawing (by pump, pressure, fluid flow, or gravity) polyolefin powder off of a reactor and melt mixing it (optionally in an extruder)—without prior pelletization of the polyolefin powder—with an adhesive graft (also called a graft (polyolefin/elastomer) copolymer) to form an adhesive resin, which is then pelletized.

The adhesive graft (also called a graft (polyolefin/elastomer) copolymer) may be pelletized separately from (and optionally prior to) the in-line process. In other words, the single heat history of the in-line process refers to the melt history of the olefin based polymer and does not include the formation (or melt history) of the adhesive graft (also called a graft (polyolefin/elastomer) copolymer).

In embodiments of in-line processes, virgin polyolefin powder may be melt mixed with the adhesive graft; and optionally, additives are introduced to the polyolefin powder before it is melt mixed with the adhesive graft. In some embodiments associated with in-line processes, the virgin polyolefin (or polyolefin stabilized with additives) may undergo cooling as it is transported from the reactor to the melt mixer; alternatively the cooling is minimized (by, for example, insulating the pipes, or using a relatively short distance of pipe—as is practical within a commercial chemical plant). In alternative embodiments of in-line processes, the virgin polyolefin (or polyolefin stabilized with additives) is stored in a vessel (such as a silo) before it is melt mixed with the adhesive graft. In this alternative embodiment, the virgin polyolefin (or polyolefin stabilized with additives) is allowed to cool more significantly and optionally to ambient or near ambient temperature.

In an alternative embodiment, an in-line process is a closed, continuous, and/or connected process for melt mixing polyolefin powder with an adhesive graft to form an adhesive resin. In one or more embodiments, a closed system is one with minor exposure to oxygen. It is to be noted that closed systems may inevitably include the exposure to oxygen either through the external introduction of oxygen and/or oxygen containing compounds to the system, leaks in pipes, via in situ generation of oxygen containing compounds within the system, or via minor amounts of oxygen that may be introduced to the reactor (for example, oxygen may be used as a catalyst terminator in the reactor) and carried through to the melt mixer (also called extruder). However, such oxygen levels are at “minor” levels such that detrimental effect/degradation is not observed in the olefin based polymer. In one or more embodiments, a connected system is one in which the olefin based polymer is manufactured and extruded on-site without the need for being moved (for example, by truck or rail) to another compounding facility (for example, a toll compounder or a compounding facility located onsite). In an embodiment, continuous and connected systems are those in which the polyolefin is carried (optionally directly) from the reactor to the melt mixer without an intermediate transportation step (by, for example, rail or truck) to a separate facility. In an embodiment, continuous and connected systems may include some intermittent storage of the polyolefin in a vessel or silo.

The in-line system is in contrast to an “off-line” system, wherein, in one or more embodiments, the olefin based polymer is produced and pelletized on one plant site. The pelletized polyolefin is then moved (optionally by truck or rail) to a second location for compounding with a graft (polyolefin/elastomer) copolymer. The second location can be a new toll compounder (i.e., a new company) or can be a separate part of a single plant site. Thus, an in-line system may utilize a single extruder, whereas an off-line system utilizes multiple extruders. As mentioned above and in various embodiments, in both the in-line and off-line processes the graft (polyolefin/elastomer) copolymer is pelletized separately (optionally in a prior system).

The in-line processes of the embodiments herein result in polyolefin based adhesive compositions exhibiting improved properties, such as reduced yellowness and/or gels, in comparison to off-line systems. Visually, yellowness is associated with product degradation by light, chemical exposure and processing. The yellowness index is calculated by the Hunter colorimeter per ASTM method E-313.

The polyolefin based adhesive composition may include the graft (polyolefin/elastomer) copolymer in a range of 0.5 wt. % to 50 wt. %, or 1 wt. % to 20 wt. %, or 2 wt. % to 15 wt. %, or 5 wt. % to 15 wt. %, or 6 wt. % to 11 wt. %, or 12 wt. % to 17 wt. %, or 20 wt.% to 30 wt.%, based on the total weight of the polyolefin based adhesive composition, for example.

In one or more embodiments, the polyolefin based adhesive composition may contain additives to impart desired physical properties, such as printability, increased gloss, or a reduced blocking tendency. Examples of additives may include, without limitation, stabilizers, ultra-violet screening agents, oxidants, anti-oxidants, anti-static agents, ultraviolet light absorbents, fire retardants, processing oils, mold release agents, coloring agents, pigments/dyes, fillers or combinations thereof, for example. These additives may be included in amounts effective to impart desired properties.

It is further contemplated that the additives may include one or more adhesion-promoting resins, such as thermoplastic elastomers.

In one or more embodiments, the additives are melt blended with the olefin based polymer and the graft (polyolefin/elastomer) copolymer. Such melt blending may occur when the olefin based polymer is melt blended with the graft (polyolefin/elastomer) copolymer, for example.

Catalyst systems useful for polymerizing olefin monomers include any suitable catalyst system. For example, the catalyst system may include chromium based catalyst systems, single site transition metal catalyst systems including metallocene catalyst systems, Ziegler-Natta (Z-N) catalyst systems or combinations thereof, for example. The catalysts may be activated for subsequent polymerization and may or may not be associated with a support material, for example. A brief discussion of such catalyst systems is included below, but is in no way intended to limit the scope of the disclosure to such catalysts.

Catalyst systems useful for polymerizing olefin monomers may include Ziegler-Natta catalyst systems, for example. Ziegler-Natta catalyst systems are generally formed from the combination of a metal component (e.g., a potentially active catalyst site) with one or more additional components, such as a catalyst support, a co-catalyst and/or one or more electron donors, for example.

A specific example of a Ziegler-Natta catalyst includes a metal component generally represented by the formula:

MRₓ;

wherein M is a transition metal, R is a halogen, an alkoxy, or a hydrocarboxyl group and x is the valence of the transition metal. For example, x may be from 1 to 4.

The transition metal may be selected from Groups IV through VIB (e.g., titanium, chromium or vanadium) of the Periodic Table of Elements, for example. R may be selected from chlorine, bromine, carbonate, ester, or an alkoxy group in various embodiments. Examples of catalyst components include TiCl₄, TiBr₄, Ti(OC₂H₅)₃Cl, Ti(OC₃H₇)₂Cl₂, Ti(OC₆H₁₃)₂Cl₂, Ti(OC₂H₅)₂Br₂ and Ti(OC₁₂H₂₅)Cl₃, for example.

Those skilled in the art will recognize that a catalyst may be “activated” in some way before it is useful for promoting polymerization. As discussed further below, activation may be accomplished by contacting the catalyst with an activator, which is also referred to in some instances as a “co-catalyst”. Embodiments of such Z-N activators include organoaluminum compounds, such as trimethyl aluminum (TMA), triethyl aluminum (TEAl) and triisobutyl aluminum (TiBAl), for example.

The Ziegler-Natta catalyst system may further include one or more electron donors, such as internal electron donors and/or external electron donors. The internal electron donors may include amines, amides, esters, ketones, nitriles, ethers, thioethers, thioesters, aldehydes, alcoholates, salts, organic acids, phosphines, diethers, succinates, phthalates, malonates, maleic acid derivatives, dialkoxybenzenes or combinations thereof, for example.

In one or more embodiments, the internal donor includes a C₃-C₆ cyclic ether, or a C₃-C₅ cyclic ether. For example, the cyclic ethers may be selected from tetrahydrofuran, dioxane, methyltetrahydrofuran and combinations thereof. (See, WIPO Pat. App. Pub. No. WO 2012/025379, which is incorporated by reference herein.)

The external electron donors may include monofunctional or polyfunctional carboxylic acids, carboxylic anhydrides, carboxylic esters, ketones, ethers, alcohols, lactones, organophosphorus compounds and/or organosilicon compounds. In one embodiment, the external donor may include diphenyldimethoxysilane (DPMS), cyclohexylmethyldimethoxysilane (CMDS), diisopropyldimethoxysilane (DIDS) and/or dicyclopentyldimethoxysilane (CPDS), for example. The external donor may be the same or different from the internal electron donor used. However, in one or more embodiments, the catalyst system is absent external donor.

The components of the Ziegler-Natta catalyst system (e.g., catalyst, activator and/or electron donors) may or may not be associated with a support, either in combination with each other or separate from one another. In one or more embodiments, the Z-N support materials may include a magnesium dihalide, such as magnesium dichloride or magnesium dibromide or silica, for example.

In one or more embodiments, the support may include a magnesium compound represented by the general formula:

MgCl₂(R″OH)ₘ;

wherein R″ is a C₁-C₁₀ alkyl and m is in a range of 0.5 to 3.

In one or more embodiments, the Ziegler-Natta catalyst system exhibits a molar ratio of support to metal component (measured as the amount of metal of each component) Mg:Ti of greater than 5:1, or in a range of 7:1 to 50:1, or 10:1 to 25:1, for example.

In one or more embodiments, the Ziegler-Natta catalyst system exhibits a molar ratio of support to internal donor Mg:ID of less than 3:1, or less than 2.9:1, or less than 2.6:1, or less than 2.1:1, or less than 2:1, or from 1.1:1 to 1.4:1, for example.

In one or more embodiments, the Ziegler-Natta catalyst system exhibits an X-ray diffraction spectrum in which the range of 2θ diffraction angles between 5.0° and 20.0°, at least three main diffraction peaks are present at diffraction angles 2θ of 7.2±0.2°, and 11.5±0.2° and 14.5±0.2°, the peak at 2θ of 7.2±0.2° being the most intense peak and the peak at 11.5±0.2° having an intensity less than 0.9 times the intensity of the most intense peak.

In one or more embodiments, the intensity of the peak at 11.5° has an intensity less than 0.8 times the intensity of the diffraction peak at 2θ diffraction angles of 7.2±0.2°. In one or more embodiments, the intensity of the peak at 14.5±0.2° is less than 0.5 times, or less than 0.4 times the intensity of the diffraction peak at 2θ diffraction angles of 7.2±0.2°.

In one or more embodiments, another diffraction peak is present at diffraction angles 2θ of 8.2±0.2° having an intensity equal to or lower than the intensity of the diffraction peak at 2θ diffraction angles of 7.2±0.2°. For example, the intensity of the peak at diffraction angles 2θ of 8.2±0.2° is less than 0.9, or less than 0.5 times the intensity of the diffraction peak at 2θ diffraction angles of 7.2±0.2°.

In one or more embodiments, an additional broad peak is observed at diffraction angles 2θ of 18.2±0.2° having an intensity less than 0.5 times the intensity of the diffraction peak at 2θ diffraction angles of 7.2±0.2°. As referenced herein, the X-ray diffraction spectra are collected by using a Bruker D8 advanced powder diffractometer.

The Ziegler-Natta catalyst may be formed by any method known to one skilled in the art. For example, the Ziegler-Natta catalyst may be formed by contacting a transition metal halide with a metal alkyl or metal hydride. (See, U.S. Pat. Nos. 4,298,718; 4,298,718; 4,544,717; 4,767,735; and 4,544,717; which are incorporated by reference herein.)

Olefin based polymers formed by catalyst systems having the specific internal donors discussed herein have been found to exhibit low levels of xylene solubles. Xylene solubles refers to the portion of a polymer that is soluble in xylene and that portion is thus termed the xylene soluble fraction (XS %). In determining XS %, the polymer is dissolved in boiling xylene and then the solution is cooled to 0° C. The XS % is that portion of the dissolved polymer that remains soluble in the cold xylene.

In one or more embodiments, the olefin based polymer exhibits a xylene soluble fraction (determined in accordance with ASTM D-5492-98) of less than 1.5%, or less than 1.0%, or less than 0.5%, for example.

Gels can originate from a number of sources, including crosslinking reactions during polymerization, insufficient mixing, homogenization during melt blending and homogenization and crosslinking during film extrusion, for example. Gels are generally undesirable as they can negatively affect subsequent film performance and appearance. For example, high concentrations of gels may cause the film to break in the film production line or during subsequent stretching. As used herein, “gels” are defined as particles having a size greater than 200 μm.

In one or more embodiments, the olefin based polymer exhibits a gel defect area of 25 ppm or less, or 20 ppm or less, for example. As used herein “gel defect area” refers to the measurement of gels in films and is measured via commercially available gel measurement systems commercially available by Optical Control Systems (OCS) GmbH, the Optical Control Systems film scanning system FS-5.

As indicated elsewhere herein, the catalyst systems are used to form olefin based polymer compositions (which may be interchangeably referred to herein as polyolefins). Once the catalyst system is prepared, as described above and/or as known to one skilled in the art, a variety of processes may be carried out using that composition to form olefin based polymers. The equipment, process conditions, reactants, additives and other materials used in polymerization processes will vary in a given process, depending on the desired composition and properties of the polymer being formed. Such processes may include solution phase, gas phase, slurry phase, bulk phase, high pressure processes or combinations thereof, for example.

In certain embodiments, the processes described above generally include polymerizing one or more olefin monomers to form olefin based polymers. The olefin monomers may include C₂ to C₃₀ olefin monomers, or C₂ to C₁₂ olefin monomers (e.g., ethylene, propylene, butene, pentene, 4-methyl-1-pentene, hexene, octene and decene), for example. It is further contemplated that the monomers may include olefinic unsaturated monomers, C₄ to C₁₈ diolefins, conjugated or nonconjugated dienes, polyenes, vinyl monomers and cyclic olefins, for example. Non-limiting examples of other monomers may include norbornene, norbornadiene, isobutylene, isoprene, vinylbenzylcyclobutane, styrene, alkyl substituted styrene, ethylidene norbornene, dicyclopentadiene and cyclopentene, for example. The formed polymer may include homopolymers, copolymers or terpolymers, for example.

The olefin based polymers may include, but are not limited to, linear low density polyethylene, elastomers, plastomers, high density polyethylenes, low density polyethylenes, medium density polyethylenes, polypropylene and polypropylene copolymers, for example.

Unless otherwise designated herein, all testing methods are the current methods at the time of filing. In one or more embodiments, the olefin based polymers include ethylene based polymers. As used herein, the term “ethylene based” is used interchangeably with the terms “ethylene polymer” or “polyethylene” and refers to a polymer having at least 50 wt. %, or at least 70 wt. %, or at least 75 wt. %, or at least 80 wt. %, or at least 85 wt. % or at least 90 wt. % polyethylene relative to the total weight of polymer, for example.

The ethylene based polymers may include one or more co-monomers, such as those discussed previously herein. For example, the ethylene based polymers may include one or more co-monomers selected from propylene, 1-butene, 1-hexene, 1-octene and combinations thereof. In one or more embodiments, the ethylene based polymer includes one or more co-monomers selected from 1-butene, 1-hexene and combinations thereof. The ethylene based polymer may include co-monomer in a range of 5 wt. % to 10 wt. %, based on the total weight of the olefin based polymer.

The ethylene based polymers may have a density (determined in accordance with ASTM D-792) of from 0.86 g/cc to 0.94 g/cc, or from 0.91 g/cc to 0.94 g/cc, or from 0.915 g/cc to 0.935 g/cc, for example.

The ethylene based polymers may have a melt index (MI₂) (determined in accordance with ASTM D-1238) of from 0.1 dg/min to 15 dg/min, from 0.1 dg/min to 10 dg/min, or from 0.05 dg/min to 8 dg/min.

In one or more embodiments, the olefin based polymers include high density polyethylene. As used herein, the term “high density polyethylene” refers to ethylene based polymers having a density of from about 0.94 g/cm³ to about 0.97 g/cm³.

In one or more embodiments, the olefin based polymers include low density polyethylene. As used herein, the term “low density polyethylene” refers to ethylene based polymers having a density in a range of 0.880 g/cm³ to 0.925 g/cm³.

In one or more embodiments, the olefin based polymers include linear low density polyethylene. As used herein, the term “linear low density polyethylene” refers to substantially linear low density polyethylene characterized by the absence of long chain branching.

In one or more embodiments, the olefin based polymers include medium density polyethylene. As used herein, the term “medium density polyethylene” refers to ethylene based polymers having a density of from 0.92 g/cm³ to 0.94 g/cm³ or from 0.926 g/cm³ to 0.940 g/cm³.

The polyolefin based adhesive compositions are useful in applications known to one skilled in the art to be useful for conventional polymeric compositions, such as forming operations (e.g., film, sheet, pipe and fiber extrusion and co-extrusion as well as blow molding, injection molding and rotary molding). Films include blown, oriented or cast films formed by extrusion or co-extrusion or by lamination useful as shrink film, cling film, stretch film, sealing films, oriented films, snack packaging, heavy duty bags, grocery sacks, baked and frozen food packaging, medical packaging, industrial liners, and membranes, for example, in food-contact and non-food contact applications. Fibers include slit-films, monofilaments, melt spinning, solution spinning and melt blown fiber operations for use in woven or non-woven form to make sacks, bags, rope, twine, carpet backing, carpet yarns, filters, diaper fabrics, medical garments and geotextiles, for example. Extruded articles include medical tubing, wire and cable coatings, sheets such as thermoformed sheets (including profiles and plastic corrugated cardboard), geomembranes and pond liners. Molded articles include single and multi-layered constructions in the form of bottles, tanks, large hollow articles, rigid food containers and toys.

In some embodiments, the homogeneous distribution of co-monomer in and among the polymer chains is important for subsequent film production. Thus, the polyolefin composition may generally exhibit a substantially homogeneous co-monomer distribution.

The polyolefin based adhesive composition can be utilized in the production of composite structures, e.g., multi-layer films, wherein a layer of the polyolefin based adhesive composition is applied to one or more layers of the multi-layer film by methods known in the art, such as co-extrusion, for example. The multi-layer films may include one or more layers formed from nylon, polyolefins, polar substrates such as ethylene vinyl alcohol (EVOH) and polyamides with one or more styrene polymers, including styrene homopolymers, copolymers, and impact modified polystyrenes. The polyolefin based adhesive compositions may be utilized as tie-layers in the multi-layer films. Tie-layers are generally utilized as a layer disposed between two additional layers to improve the adhesion therebetween.

Tie-layers in the composite structures may experience significant stresses which are created at an interface between the tie-layer and the layer to which the tie-layer is adhered. However, the tie-layer adhesives of the embodiments described herein exhibit substantial and unexpected adhesive properties even under significant stresses.

The multi-layer film may include any number of layers sufficient to satisfy its application. For example, the multi-layer film may include at least 2, or 3, or 4, or 5 or 6, or 7, or 9, or 11 layers.

The polyolefin based adhesive compositions disclosed herein exhibit excellent adhesion under a variety of conditions to non-polar polyolefins, polar polymers and styrenic substrates.

### EXAMPLES

To facilitate a better understanding of the disclosure, the following examples of embodiments are given. In no way should the following examples be read to limit, or to define, the scope of the appended claims.

An adhesive composition was evaluated for use as tie-layers in multi-layer films. The adhesive composition included about 73.5 wt. % ethylene hexene LLDPE and about 26.5 wt. % graft (polyolefin/elastomer) copolymer and exhibited a density of about 0.922 g/cm³. The LLDPE was prepared with the Ziegler-Natta catalyst described herein.

The graft (polyolefin/elastomer) copolymer was a graft of (a) a live graft of high density polyethylene grafted with maleic anhydride and (b) elastomer comprising ethylene-propylene rubber (EPR). The graft (polyolefin/elastomer) copolymer had a maleic anhydride content of 1.5 wt. %.

The in-line samples were prepared via a single heat cycle by discharging a polyolefin from a polymerization reactor in the form of a powder and feeding the polyolefin into an accumulator bin in-line with the reactor. The graft (polyolefin/elastomer) copolymer was introduced into a second accumulator bin and then both components were fed together into a mixer where they were mixed and heated to a temperature of about 400-450° F. (204.4-232.2° C.), subjected to shear mixing and pelletized. The off-line samples were prepared via multiple heat cycles (e.g., previously manufactured and pelletized resin mixed with graft (polyolefin/elastomer) copolymer in a twin screw extruder heated to a temperature of about 400-450° F. (204.4-232.2° C.), subjected to shear mixing and pelletized).

To determine the gel counts and distribution of gels in the various adhesive compositions, samples of each adhesive composition were separately introduced into a single screw extruder and extruded into 2 mil monolayer cast films. The content of gelled polymer in the resulting films was determined by counting the number of gels in a given area (10 m²) and normalizing the count by a laser gel scanner (i.e., film inspection methods commercially available through OCS (Optical Control Systems) GmbH). The results are illustrated in TABLE 1 and TABLE 2 below:

TABLE 1

| Inventive | Gel | Comparative | Gel Area | Ratio of Gel Area |
|---|---|---|---|---|
| Examples | Area (ppm) | Examples | (ppm) | (Off-line to In-line) |
| In-line-1 | 30 | Off-line-1 | 27 | 0.88 |
| In-line-2 | 30 | Off-line-2 | 45 | 1.48 |
| In-line-3 | 21 | Off-line-3 | 39 | 1.89 |
| In-line-4 | 20 | Off-line-4 | 56 | 2.80 |
| In-line-5 | 24 | Off-line-5 | 36 | 1.48 |
| In-line-6 | 25 | Off-line-6 | 39 | 1.56 |
| In-line-7 | 28 | Off-line-7 | 37 | 1.30 |
| In-line-8 | 23 | Off-line-8 | 37 | 1.58 |
| Average | 25 | Average | 39 | 1.56 |

TABLE 2

| | | | | Ratio of Total |
|---|---|---|---|---|
| | | | Total | Number of Gels |
| Inventive | | Comparative | Gel | (Off-line to |
| Examples | Total Gel Count | Examples | Count | In-line) |
| In-line-1 | 324 | Off-line-1 | 289 | 0.89 |
| In-line-2 | 440 | Off-line-2 | 442 | 1.01 |
| In-line-3 | 253 | Off-line-3 | 388 | 1.53 |
| In-line-4 | 241 | Off-line-4 | 520 | 2.16 |
| In-line-5 | 321 | Off-line-5 | 362 | 1.13 |
| In-line-6 | 327 | Off-line-6 | 383 | 1.17 |
| In-line-7 | 374 | Off-line-7 | 371 | 0.99 |
| In-line-8 | 299 | Off-line-8 | 371 | 1.24 |
| Average | 322 | Average | 391 | 1.21 |

It was observed that the adhesive compositions prepared by the embodiments described herein, exhibited gel counts substantially lower than that of the adhesive compositions prepared via off-line methods.

The yellowness index of various in-line samples was further measured via ASTM method E-313 and is reported in TABLE 3 below:

TABLE 3

| | | | Yellow | |
|---|---|---|---|---|
| | | | Index | |
| Inventive | Yellow Index | Comparative | D1925 | Ratio of YI |
| Examples | D1925 (Index) | Examples | (Index) | (Off-line to In-line) |
| In-line-1 | 1.70 | Off-line-1 | 5.49 | 3.23 |
| In-line-2 | 1.10 | Off-line-2 | 5.96 | 5.42 |
| In-line-3 | 0.70 | Off-line-3 | 5.80 | 8.29 |
| In-line-4 | 0.30 | Off-line-4 | 5.70 | 19 |
| In-line-5 | 0.70 | Off-line-5 | 5.92 | 8.46 |
| In-line-6 | 1.20 | Off-line-6 | 6.09 | 5.08 |
| In-line-7 | 0.01 | Off-line-7 | 6.12 | 612 |
| In-line-8 | 0.01 | Off-line-8 | 5.80 | 580 |
| Average | 1 | Average | 6 | 6 |

While the foregoing is directed to embodiments of the present disclosure, further embodiments of the disclosure may be devised without departing from the scope of the present disclosure.

What is claimed is:

1. A process of forming an adhesive composition comprising:
contacting an olefin monomer with a catalyst system within a polymerization zone to form an olefin based polymer under polymerization conditions sufficient to form the olefin based polymer, the catalyst system comprising a metal component generally represented by the formula:
MRₓ;
wherein M is a transition metal, R is a halogen, an alkoxy, or a hydrocarboxyl group and x is the valence of the transition metal, wherein the catalyst system further comprises an internal donor (ID) comprising a C₃-C₆ cyclic ether; and
withdrawing the olefin based polymer from the polymerization zone; and
melt blending the olefin based polymer with a graft (polyolefin/elastomer) copolymer to form a polyolefin based adhesive composition,
wherein the process is an in-line process.

2. The process of claim 1, wherein the olefin based polymer contacts the graft (polyolefin/elastomer) copolymer prior to pelletization of the olefin based polymer.

3. The process of claim 1, further comprising melt blending the olefin based polymer and the graft (polyolefin/elastomer) copolymer in the presence of an adhesion promoting additive.

4. The process of claim 1, wherein the transition metal is selected from titanium, chromium and vanadium.

5. The process of claim 1, wherein the metal component is selected from TiCl₄, TiBr₄, Ti(OC₂H₅)₃Cl, Ti(OC₃H₇)₂Cl₂, Ti(OC₆H₁₃)₂Cl₂, Ti(OC₂H₅)₂Br₂ and Ti(OC₁₂H₂₅)Cl₃.

6. The process of claim 1, wherein the catalyst system further comprises an organoaluminum compound selected from trimethyl aluminum (TMA), triethyl aluminum (TEAl) and triisobutyl aluminum (TiBAl).

7. The process of claim 1, wherein the cyclic ethers are selected from tetrahydrofuran, dioxane, methyltetrahydrofuran and combinations thereof.

8. The process of claim 1, wherein the catalyst system further comprises a support material comprising a magnesium halide.

9. The process of claim 8, wherein the catalyst system exhibits a molar ratio Mg:Ti of greater than 5:1.

10. The process of claim 8, wherein the catalyst system exhibits a molar ratio Mg:ID of less than 3:1.

11. The process of claim 1, wherein the olefin based polymer comprises polyethylene.

12. The process of claim 11, wherein the olefin based polymer exhibits a density (determined in accordance with ASTM D-792) of from 0.86 g/cm³ to 0.94 g/cm³.

13. The process of claim 11, wherein the olefin based polymer exhibits a melt index (MI₂) (determined in accordance with ASTM D-1238) in a range of 0.1 dg/min to 15 dg/min.

14. The process of claim 1, wherein the graft (polyolefin/elastomer) copolymer comprises a functional monomer selected from carboxylic acids and carboxylic acid derivatives, and acid and acid anhydride derivatives.

15. The process of claim 1, wherein the polyolefin based adhesive composition comprises the graft (polyolefin/elastomer) copolymer in a range of 0.5 wt. % to 50 wt. %, based on the total weight of the polyolefin based adhesive composition.

16. An adhesive composition comprising:
a polyolefin based adhesive composition formed with a single heat cycle and comprising:
an olefin based copolymer of ethylene and a co-monomer selected from propylene, 1-butene, 1-hexene, 1-octene and combinations thereof formed with catalyst system comprising a metal component generally represented by the formula:
MRₓ;
wherein M is a transition metal, R is a halogen, an alkoxy, or a hydrocarboxyl group and x is the valence of the transition metal, wherein the catalyst system further comprises an internal donor comprising a C₃-C₆ cyclic ether; and supported on MgCl₂; and
a graft (polyolefin/elastomer) copolymer.

17. The adhesive composition of claim 16, wherein the composition comprises a lower gel count and a lower yellowness index than an identical composition formed via an off-line process.

18. The process of claim 1, wherein the cyclic ethers are selected from tetrahydrofuran, dioxane, methyltetrahydrofuran and combinations thereof.

19. A multi-layer film comprising:
a plurality of resin layers; and
one or more tie-layers disposed between at least two of the resin layers, wherein the tie layers are formed of the adhesive composition of claim 16.
